(19) United States
(12) Patent Application Publication
(10) Pub. No.: US 2023/0001178 A1
(43) Pub. Date: Jan. 5, 2023

(54) HEART PUMP ASSEMBLY WITH A BLOOD INLET CONFIGURED TO INCREASE BLOOD FLOW

(71) Applicant: ABIOMED, Inc., Danvers, MA (US)

(72) Inventors: Scott C. Corbett, Danvers, MA (US); Zhongwei Qi, Danvers, MA (US)

(73) Assignee: ABIOMED, Inc., Danvers, MA (US)

(21) Appl. No.: 17/852,645

(22) Filed: Jun. 29, 2022

Related U.S. Application Data
(60) Provisional application No. 63/217,575, filed on Jul. 1, 2021.

(51) Int. Cl. A61M 60/13; A61M 60/216

(52) U.S. Cl. CPC A61M 60/13; A61M 60/216

(57) ABSTRACT

A heart pump assembly having a blood inlet configured to increase blood flow into the heart pump assembly is disclosed herein. The heart pump assembly includes a motor housing, a cannula connected to the motor housing, and a blood inlet connected to the cannula. The blood inlet has a distal body portion, a proximal body portion defining an inlet conduit therewithin, and a plurality of cage openings defined and positioned between the distal and proximal body portions. The inlet conduit has one of a tapered portion, a frustrum-shaped portion, or both a tapered portion and a frustrum-shaped portion and is adapted to reduce flow turbulence at the blood inlet and increase the blood flow into the heart pump.

### CROSS REFERENCE TO RELATED APPLICATION

This application claims the benefit of the U.S. Provisional Application No. 63/217,575, which was filed on Jul. 1, 2021 and is incorporated herein by reference.

### TECHNICAL FIELD

The present invention relates to heart pump assembly, and more particularly, to heart pump assembly having a blood inlet for increasing blood flow into the heart pump assembly.

### BACKGROUND OF THE INVENTION

A heart pump, such as a percutaneous intracardiac heart pump assembly, can be inserted into a heart to deliver blood from the heart into an artery. When deployed in the heart, a heart pump assembly pulls blood from the left ventricle of the heart and expels blood into the aorta, or pulls blood from the right ventricle and expels blood into the pulmonary artery. Specifically, the blood enters the heart pump assembly via a blood inlet located at a distal end of the heart pump assembly, travels through a cannula of the heart pump assembly, and expels via a plurality of apertures defined at a proximal end of the heart pump assembly. However, the design of the currently available blood inlet of a heart pump assembly produces large blood flow recirculation at the entry (or inlet) of the blood inlet, thereby reducing blood flow into the heart pump assembly.

Accordingly, there exists a need for a blood inlet used with a heart pump assembly to prevent large blood recirculation at the entry to maximize the blood flow into the heart pump assembly.

### BRIEF SUMMARY OF THE INVENTION

Described herein is a heart pump assembly having a blood inlet. The heart pump assembly includes a motor housing, a cannula connected to the motor housing, and a blood inlet connected to the cannula. The blood inlet has a distal body portion, a proximal body portion defining an inlet conduit therewithin, and a plurality of cage openings defined and positioned between the distal and proximal body portions. The inlet conduit has one of a tapered portion, a frustrum-shaped portion, or both a tapered portion and a frustrum-shaped portion and is adapted to reduce flow turbulence at the blood inlet and increase the blood flow into the heart pump.

These and other aspects of the present invention will be better understood in view of the drawings and following detailed description.

### BRIEF DESCRIPTION OF DRAWINGS

FIG. 1 is a perspective view of a heart pump assembly, according to an embodiment of the present invention.

FIG. 2 is a side perspective view of the blood inlet of the heart pump assembly of FIG. 1.

FIG. 3 is a side cross-sectional view of the blood inlet of FIG. 2 along line 3-3.

FIG. 4 is a side cross-sectional view of an exemplary prior art blood inlet of a heart pump assembly.

FIG. 5 shows blood flow recirculation at the entry (or inlet) of the blood inlet of FIG. 4.

FIG. 6 shows blood flow recirculation at the entry (or inlet) of the blood inlet of FIG. 2.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The heart pump assembly may be percutaneously inserted into the heart through the aorta. The blood inlet may be positioned past the aortic valve in the left ventricle, in order to pull blood from the left ventricle and expel the blood into the aorta. Although the atraumatic tip spaces the heart pump assembly from the heart walls, in some instances, the blood inlet may be positioned near to the walls of the heart or various heart structures, such as the leaflets of the mitral valve. The heart pump assembly described herein provides a heart pump assembly with an improved blood inlet. The blood inlet is configured and designed to prevent or reduce blow flow resistance and blood flow recirculation at the entry of the blood inlet, thereby increasing blood flow into the heart pump assembly, as will be described in detail below.

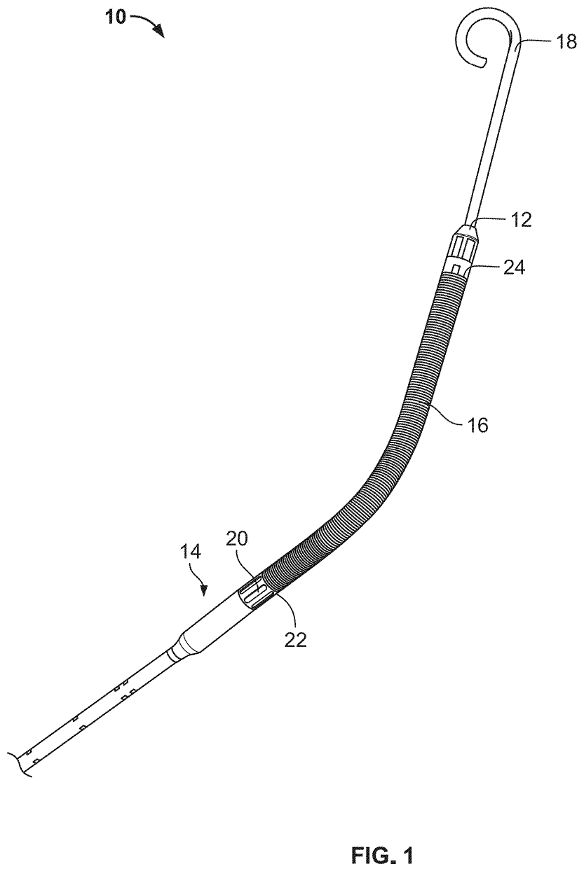

FIG. 1 illustrates a heart pump 10 assembly comprising an improved blood inlet 12 in accordance with the present technology. The heart pump assembly 10 includes a motor housing 14, a cannula 16, and an atraumatic tip 18 for stabilizing the heart pump assembly 10 in the ventricle of the heart. The heart pump assembly 10 can vary in any number of ways. For example, the embodiment of FIG. 1 herein may exclude a motor housing. Instead, the motor can be configured to be positioned outside of a patient's body and can operatively couple to the rotor via a drive shaft or cable.

The motor housing 14 is configured to accommodate an impeller (not shown) and a motor (not shown) therewithin. The motor is used to rotate the impeller to draw blood from the heart into the heart pump assembly 10. Specifically, rotation of the blades of the impeller creates suction through the cannula 16 for the blood to flow into the heart pump assembly 10. The blood enters the cannula 16 and travels therethrough and exits the heart pump assembly 10 from a plurality of blood exhaust outlets 20 defined adjacent to or on the motor housing 14.

Referring again to FIG. 1, the cannula 16 extends between a proximal end 22 and a distal end 24. At the distal end 24 of the cannula 16, the cannula 16 and blood inlet 12 are connected and are in fluid communication with each other. The cannula 16 is also connected to the motor housing 14 at the proximal end 22 thereof. The blood inlet 12 is also connected to the atraumatic tip 18. Thus, the blood inlet 12, cannula 16, motor housing 14, and atraumatic tip 18 are all connected and are all in fluid communication with each other.

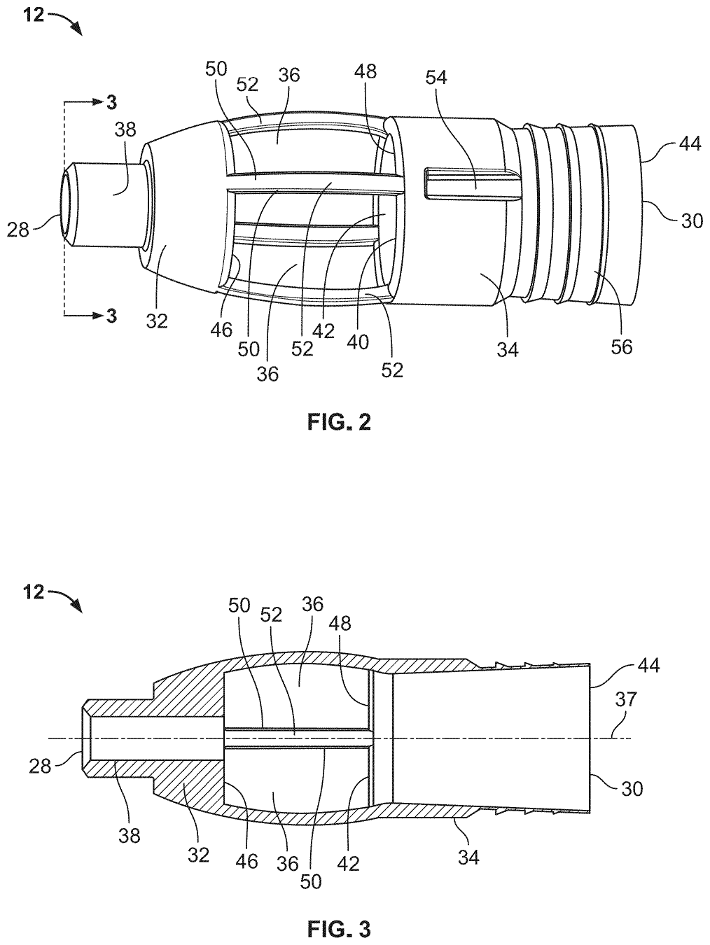

Referring to FIGS. 2 and 3, the blood inlet 12 extends between a distal end 28 and a proximal end 30, and includes a distal body portion 32, a proximal body portion 34, and a plurality of cage openings 36 defined and positioned between the distal and proximal body portions 32, 34. The distal body portion 32 of the blood inlet 12 includes a connector 38 for connecting the atraumatic tip 18 (shown in FIG. 1) to the blood inlet 12 at its distal end 28.

The atraumatic tip 18 may be shaped as a flexible extension having a pigtail as shown in FIG. 1. Alternatively, the atraumatic tip 18 is configured as a straight extension or as a ball. Also, the atraumatic tip 18 may include a lumen for the passage of a guidewire through the atraumatic tip 18. The atraumatic tip 18 acts as a mechanical spacer that provides a space between the plurality of cage openings 36 of the blood inlet 12 and the inner surface of the heart. This space prevents the plurality of cage openings 36 from suctioning to the walls of the heart, heart valves (e.g., the mitral valve), or any other anatomical structure in the heart. This can reduce the risk of blockage of the plurality of cage openings 36 and may reduce or prevent damage to the heart tissues.

The proximal body portion 34 defines an inlet conduit 40 therewithin for the drawn blood to travel therethrough and into the cannula 16. The inlet conduit 40 of the proximal body portion 34 of the blood inlet 12 extends between a first open end 42 of the proximal body portion 34 and a second open end 44 of the proximal body portion 34. As shown in FIG. 3, the inlet conduit 40 is frustrum-shaped. Thus, the inlet conduit 40 has a first inner diameter at the first open end 42 that is less than a second inner diameter at the second open end 44. The blood inlet 12 having a tapered inlet conduit 40 reduces the flow resistance of the blood at the inlet of the proximal body portion 34 of the blood inlet 12 as the blood enters the heart pump assembly 10 (shown in FIG. 1), thereby increasing the blood flow. The first inner diameter at the first open end 42 and the second inner diameter at the second open end 44 are approximately 3.5 cm and 4.1 cm, respectively.

Referring again to FIGS. 2 and 3, the plurality of cage openings 36 are oriented radially around a circumference of the blood inlet 12 with an evenly spaced distance between each of the plurality of cage openings 36. Each of the plurality of cage openings 36 has an associated height measured parallel to a longitudinal axis 37, a width measured transverse to the longitudinal axis 37, and an area. For example, each cage opening 36 has a height, a width, and an area through which blood may enter and flow through the cannula 16 (shown in FIG. 1) from the inlet of the blood inlet 12. In one embodiment, each of the plurality of cage openings 36 has identical measurements (e.g., height, width, and area) such that the cage openings 36 are also substantially identical.

In the illustrated embodiment, each of the plurality of cage openings 36 has a shape with flat distal and proximal edges 46, 48 and curved outer edges 50. The plurality of cage openings 36 may have any suitable shape to allow blood to enter the cannula 16. For example, the plurality of cage openings 36 can be oblong, oval, square, tear-shaped, round or any other suitable shape.

A valve leaflet or other portion of the anatomy that is suctioned against some of the plurality of cage openings 36, or into the heart pump assembly 10, decreases the area through which blood can enter the cannula 16, thereby potentially decreasing the flow rate of the blood through the heart pump assembly 10. Because the plurality of cage openings 36 are relatively small in size, the cage openings 36 are less likely to allow a valve leaflet to enter the heart pump assembly 10 at the inlet, allowing the interior of the cannula 16 to be clear for the passage of blood.

Each of the plurality of cage openings 36 are defined by edges. Specifically, a distal edge 46 and a proximal edge 48 of each cage opening 36 are defined by a portion of the distal body portion 32 and a portion of the proximal body portion 34 of the blood inlet 12, respectively, as shown in FIGS. 2 and 3. In addition, outer edges 50 of each of the plurality of cage openings 36 are defined by a plurality of struts 52 included in the blood inlet 12 such that each of the plurality of cage openings 36 is separated by the plurality of struts 52. In one embodiment, the distal edge 46 and the proximal edge 48 are filleted. In another embodiment, the plurality of struts 52 are filleted. In yet another embodiment, all of the distal edge 46, the proximal edge 48, and the plurality of struts 52 are filleted.

The plurality of struts 52 are connected to the distal body portion 32 and proximal body portion 34 of the blood inlet 12, as shown in FIGS. 2 and 3. In the illustrated embodiment, the plurality of struts 52 are outwardly bowed or curved. These bowed or curved struts 52 provide a smooth transition into the curve-shaped distal body portion 32 of the blood inlet 12 and the tapered proximal body portion 34 to reduce the force absorbed when it is inserted into the patient's body. The plurality of struts 52 can function as a screen to prevent the suctioning of valve leaflets and other vascular or heart tissues to the plurality of cage openings 36.

Referring again to FIG. 2, the blood inlet 12 further includes a cutoff 54 defined on an outer surface of the proximal body portion 34 of the blood inlet 12. The cutoff 54 provides a place for a sensor (not shown) to be mounted and secured thereon. In addition, a barb 56 is defined on the outer surface of the proximal body portion 34 of the blood inlet 12 for providing a tight connection and securing the cannula 16 to the blood inlet 12.

The heart pump assembly 10 is made of one or more materials having suitable properties for a desired application, including strength, weight, rigidity, etc. Plastic (e.g., polypropylene, polyethylene, etc.) is preferred for the blood inlet 12, atraumatic tip 18, and motor housing 14.

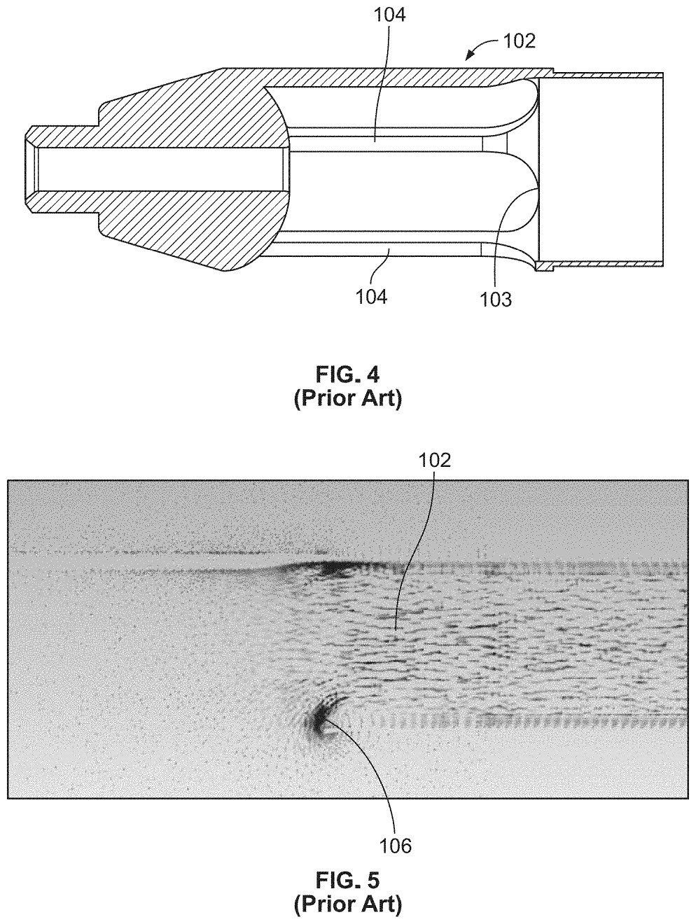

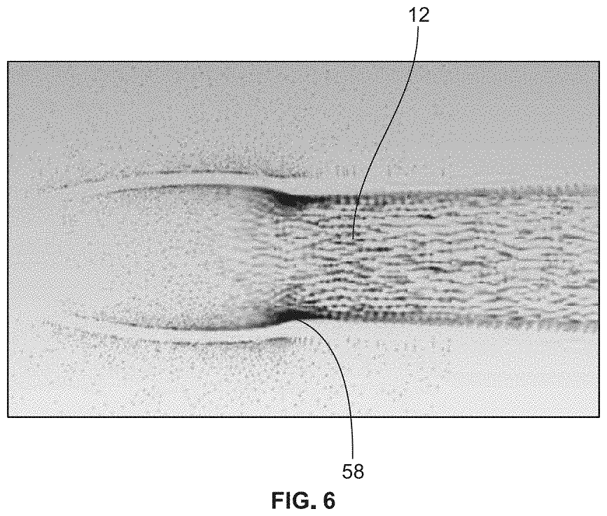

FIG. 4. is a side cross-sectional view of an exemplary prior art blood inlet 102 of a heart pump assembly. The prior art blood inlet 102 has a plurality of struts 104 that are straight, instead of bowed as are the struts 52 in the blood inlet 12 described herein. The design of the prior art blood inlet 102 produces large blood flow recirculation at the entry (inlet) 103 of the blood inlet 102, as shown at the location 106 in FIG. 5. Therefore, the blood flow at the entry of the prior art blood inlet 102 has some turbulence, illustrated as 106, caused by the transition from the blood inlet 102 to the inlet 103. As stated above, the design and construction of the blood inlet 12 of the heart pump assembly 10 in the illustrated embodiment effectively reduces the blood flow recirculation that occurs at the entry of the prior art blood inlet 102, thereby providing more laminar blood flow into the inlet conduit 40 of the blood inlet 12. Specifically, the improved design of the blood inlet 12 having a tapered contour of the blood inlet 12 and frustrum-shaped inlet conduit 40 effectively reduces flow turbulences at the blood inlet 12 and the inlet conduit 40, thereby increasing the blood flow into the heart pump assembly 10. This laminar flow at the entry of the blood inlet 12 of the heart pump assembly 10 is illustrated at the location 58 in FIG. 6.

In one aspect, described herein is a heart pump assembly having a motor housing, the motor housing including an impeller and a motor therewithin. There is a cannula connected to the motor housing and a blood inlet connected to the cannula, the blood inlet having a distal body portion, a proximal body portion, and a plurality of cage openings defined and positioned between the distal and proximal body portions, the proximal body portion defines an inlet conduit therewithin. The inlet conduit has one of a tapered portion, a frustrum-shaped portion, or both a tapered portion and a frustrum-shaped portion, adapted to reduce flow turbulence at the blood inlet and increase the blood flow into the heart pump.

In a further aspect, the heart pump assembly has an atraumatic tip extending from the distal body portion of the blood inlet for stabilizing the heart pump assembly, when placed in a patient's heart. In a further aspect, the plurality of blood exhaust outlets may be adjacent to or on the motor housing for blood to exit the heart pump assembly. In yet a further aspect, the distal body portion of the blood inlet includes a connector for connecting the atraumatic tip to the blood inlet. In any of the above aspects, the inlet conduit of the proximal body portion of the blood inlet may extend between a first open end of the proximal body portion and a second open end of the proximal body portion. In any of the above aspects, the inlet conduit has a first inner diameter at the first open end that is less than a second inner diameter at the second open end. In any of the above aspects, the plurality of cage openings may be oriented radially around a circumference of the blood inlet with an evenly spaced distance between each of the plurality of cage openings. In any of the above aspects, each of the plurality of cage openings has an associated height measured parallel to a longitudinal axis, a width measured transverse to the longitudinal axis, and an area. In any of the above aspects, the plurality of cage openings may be substantially identical in both size and shape. In the above aspects, each of the plurality of cage openings may have a bowed shape with flat distal and proximal edges and curved outer edges.

In the above aspects, the distal edge and the proximal edge of each of the plurality of cage openings may be defined by a portion of the distal body portion and a portion of the proximal body portion of the blood inlet. In a further aspect, the blood inlet further includes a plurality of struts that are connected to the distal body portion and proximal body portion of the blood inlet. The outer edges of each of the plurality of cage openings may be defined by the plurality of struts such that each of the plurality of cage openings may be separated by the plurality of struts. The plurality of struts may be outwardly bowed or curved to provide a smooth transition into the curve-shaped distal body portion of the blood inlet and the tapered proximal body portion to reduce the force absorbed when it is inserted into a patient's body.

In any of the above aspects, the blood inlet may further include a cutoff defined on an outer surface of the proximal body portion of the blood inlet. In any of the above aspects, a barb is defined on an outer surface of the proximal body portion of the blood inlet for providing a secure connection for the cannula to the blood inlet. In the above aspects, the blood inlet, cannula, motor housing, and atraumatic tip may all be connected and are all in fluid communication with each other.

In any of the above aspects, the plurality of cage openings may be oblong, oval, square, tear-shaped or round. In these aspects, the distal edge and the proximal edge of each cage opening may be filleted.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A heart pump assembly comprising:
a motor housing, the motor housing including an impeller and a motor therewithin;
a cannula connected to the motor housing; and
a blood inlet connected to the cannula, the blood inlet having a distal body portion, a proximal body portion, and a plurality of cage openings defined and positioned between the distal and proximal body portions, the proximal body portion defines an inlet conduit therewithin,
wherein the inlet conduit has one of a tapered portion, a frustrum-shaped portion, or both a tapered portion and a frustrum-shaped portion, adapted to reduce flow turbulence at the blood inlet and increase the blood flow into the heart pump assembly.

2. The heart pump assembly of claim 1, further comprising an atraumatic tip extending from the distal body portion of the blood inlet for stabilizing the heart pump assembly, when placed in a patient's heart.

3. The heart pump assembly of claim 1, wherein each of a plurality of blood exhaust outlets are located adjacent to or on the motor housing for blood to exit the heart pump assembly.

4. The heart pump assembly of claim 2, wherein the distal body portion of the blood inlet includes a connector for connecting the atraumatic tip to the blood inlet.

5. The heart pump assembly of claim 1, wherein the inlet conduit of the proximal body portion of the blood inlet extends between a first open end of the proximal body portion and a second open end of the proximal body portion.

6. The heart pump assembly of claim 5, wherein the inlet conduit has a first inner diameter at the first open end that is less than a second inner diameter at the second open end.

7. The heart pump assembly of claim 1, wherein the plurality of cage openings is oriented radially around a circumference of the blood inlet with an evenly spaced distance between each of the plurality of cage openings.

8. The heart pump assembly of claim 1, wherein each of the plurality of cage openings has an associated height measured parallel to a longitudinal axis, a width measured transverse to the longitudinal axis, and an area.

9. The heart pump assembly of claim 1, wherein each of the plurality of cage openings are substantially identical in both size and shape.

10. The heart pump assembly of claim 1, wherein each of the plurality of cage openings has a bowed shape with flat distal and proximal edges and curved outer edges.

11. The heart pump assembly of claim 10, wherein the distal edge and the proximal edge of each of the plurality of cage openings are defined by a portion of the distal body portion and a portion of the proximal body portion of the blood inlet.

12. The heart pump assembly of claim 11, wherein the blood inlet further includes a plurality of struts that are connected to the distal body portion and proximal body portion of the blood inlet.

13. The heart pump assembly of claim 12, wherein the outer edges of each of the plurality of cage openings are defined by the plurality of struts such that each of the plurality of cage openings is separated by the plurality of struts.

14. The heart pump assembly of claim 13, wherein each of the plurality of struts are outwardly bowed or curved to provide a smooth transition into a curve-shaped distal body portion of the blood inlet and the tapered portion that is a tapered proximal body portion to reduce the force absorbed when it is inserted into a patient's body.

15. The heart pump assembly of claim 1, wherein the blood inlet further includes a cutoff defined on an outer surface of the proximal body portion of the blood inlet.

16. The heart pump assembly of claim 1, wherein a barb is defined on an outer surface of the proximal body portion of the blood inlet for providing a secure connection for the cannula to the blood inlet.

17. The heart pump assembly of claim 2, wherein the blood inlet, cannula, motor housing, and atraumatic tip are all connected and are all in fluid communication with each other.

18. The heart pump assembly of claim 1, wherein each of the plurality of cage openings are oblong, oval, square, tear-shaped or round.

19. The heart pump assembly of claim 11, wherein the distal edge and the proximal edge of each cage opening are filleted.
